(19) United States
(12) Patent Application Publication
(10) Pub. No.: US 2026/0295167 A1
(43) Pub. Date: Oct. 1, 2026

(54) Readable Syringe for Dosage Verification System

(71) Applicant: Retractable Technologies, Inc., Little Elm, TX (US)

(72) Inventors: Thomas J. Shaw, Frisco, TX (US); Benjamin John Lardas, Denton, TX (US)

(21) Appl. No.: 19/635,352

(22) Filed: Mar. 31, 2026

Related U.S. Application Data
(60) Provisional application No. 63/781,026, filed on Mar. 31, 2025.

(51) Int. Cl. A61M 5/31; A61M 5/32

(52) U.S. Cl. CPC A61M 5/3129; A61M 5/3202; A61M 2205/6081

(57) ABSTRACT

Systems and methods are disclosed that include providing a readable syringe for a dosage verification system. The readable syringe includes a transparent barrel comprising a fluid chamber configured to receive a volume of fluid, a first related component that is a first solid color, and a second related component that is a second solid color opposite of the first solid color. The first related component moves relative to the second related component and is distinguishable from the second related component in a digital image captured by an imaging device to determine a position of the first related component with respect to the second related component. The imaging device utilizes known characteristics of the syringe and the position of the first related component with respect to the second related component in the digital image to accurately determine the volume of fluid in the syringe.

### CROSS-REFERENCE TO RELATED APPLICATIONS

This application claims priority under 35 U.S.C. § 119(e) to U.S. Provisional Application No. 63/781,026, filed Mar. 31, 2025 by Thomas J. Shaw, et al. and entitled “Readable Syringe for Dosage Verification System,” the disclosure of which is hereby incorporated by reference in its entirety for all purposes.

### BACKGROUND

Medical syringes are used for administering injections or infusions of drugs, medicines, and vaccines all over the world. Such injections are administered by both medical professionals and in some instances, self-administered. In both situations, dosage accuracy is crucial in preventing a medical dosing error that could have adverse effects on the patient. Dosing errors may arise from mistakes in administering or calculating the desired or recommended dosage, which may be based on patient age, patient weight, and/or blood glucose level. Dosing errors may also arise from mistakes in drawing the incorrect dosage into a syringe, and/or reading the drawn dosage on a volumetric scale of a syringe.

For smaller volume syringes, such as 1.0 mL and 0.5 mL, the barrel curvature associated with the small diameter of a conventional cylindrical syringe makes the associated markings of the volumetric scale and related numerical indicia difficult to accurately read by both medical professionals and self-administering patients. Accurately reading a drawn dose in such circumstances can be difficult for medical professionals, thereby further increasing the likelihood of dosing errors when administering injections. Such likelihood of errors is further compounded with patients or non-professional caregivers drawing up their own doses for injection or other administration.

Various protocols have been instituted in an effort to reduce such medical errors, but such errors remain a principal concern. In many healthcare facilities, a medical professional or caregiver must have another person visually verify the medicine and the drawn dosage prior to administering an injection to a patient. Such a requirement can pose an additional burden on staffing of clinics or hospitals. Accordingly, the medical industry continues to demand advances in syringe and dosage verification technology.

### SUMMARY

Embodiments of a readable syringe for a dosage verification system disclosed herein may include: a transparent barrel including a fluid chamber configured to receive a volume of fluid; a first related component that is a first solid color; a second related component that is a second solid color opposite of the first solid color; wherein the first related component moves relative to the second related component; wherein the first related component is distinguishable from the second related component by an imaging device in a digital image captured by the imaging device to determine a position of the first related component with respect to the second related component; and wherein the position of the first related component with respect to the second related component in the digital image is used by the imaging device to determine the volume of fluid in the syringe.

Embodiments of a readable syringe for a dosage verification system disclosed herein may include: a transparent barrel including a cylindrical body portion that defines a fluid chamber configured to receive a volume of fluid within the barrel, a nose disposed at a forward end of the cylindrical body portion, an annularly projecting finger flange disposed around the cylindrical body portion, a pair of substantially flat, parallel opposing indicia display surfaces that extend tangentially from the cylindrical body portion and extend forwardly from the finger flange and longitudinally along the cylindrical body portion proximally to the nose; and a cylindrical collar that extends rearwardly from the finger flange and provides a rearwardly facing opening into the fluid chamber defined by the cylindrical body portion; a plunger received through the rearwardly facing opening and including a plunger surface, a plunger body, and a plunger seal slidably engaging the fluid chamber; a needle extending from the nose; an elastomeric bushing disposed around the cylindrical collar; and a needle safety device slidably engaged with the pair of substantially flat, parallel opposing indicia display surfaces; wherein at least one of the plunger and the plunger seal include a first related component that is a first solid color; wherein at least one of the needle safety device and the elastomeric bushing include a second related component that is a second solid color opposite of the first solid color; wherein the first related component moves relative to the second related component; wherein the first related component is distinguishable from the second related component by an imaging device in a digital image captured by the imaging device to determine a position of the first related component with respect to the second related component; and wherein the position of the first related component with respect to the second related component in the digital image is used by the imaging device to determine the volume of fluid in the syringe.

Embodiments of a readable syringe for a dosage verification system disclosed herein may include: a readable syringe; and an imaging device configured to capture a digital image of a first related component and a second related component of the syringe, distinguish the first related component from the second related component in the digital image to determine a position of the first related component with respect to the second related component, and use the position of the first related component with respect to the second related component in the digital image to determine a volume of fluid in the syringe.

Embodiments of a readable syringe for a dosage verification system disclosed herein may include: a barrel comprising: a cylindrical body portion that defines a fluid chamber; a pair of substantially flat, opposing indicia display surfaces disposed in parallel and extending longitudinally along the cylindrical body portion; and an annularly projecting finger flange disposed around the cylindrical body portion, wherein the annularly projecting finger flange comprises: a first flat edge disposed in parallel to a first indicia display surface of the pair of substantially flat, opposing indicia display surfaces; a second opposing flat edge disposed in parallel to a second indicia display surface of the pair of substantially flat, opposing indicia display surfaces; a first curved edge joined to the first flat edge; and a second curved edge joined to the second flat edge; wherein the first curved edge and the second curved edge cooperate to automatically rotate the syringe to a position where one of the first flat edge and the second flat edge rest against a flat surface when the syringe is placed on the flat surface such that one of the first curved edge and the second curved edge contacts the flat surface to rotationally orient either the first indicia display surface or the second indicia display surface in an upward facing position.

Embodiments of a method of determining a volume of fluid in a syringe disclosed herein may include: providing a dosage verification system including an imaging device and a readable syringe according to any of the preceding claims; positioning the imaging device relative to the syringe; capturing a digital image of a first related component and a second related component of the syringe; processing the digital image to distinguish the first related component from the second related component in the digital image to determine a position of the first related component with respect to the second related component; and determining the volume of the fluid in the syringe based on the position of the first related component with respect to the second related component in the digital image.

### BRIEF DESCRIPTION OF THE DRAWINGS

For a more complete understanding of the present disclosure and so that the manner in which the features and advantages of the embodiments can be understood in more detail, reference is now made to the following brief description, taken in connection with the accompanying drawings and detailed description.

FIG. 1 shows an oblique view of a syringe according to an embodiment of the disclosure.

FIG. 2 shows another oblique view of the syringe according to an embodiment of the disclosure.

FIG. 3 shows an oblique exploded view of the syringe according to an embodiment of the disclosure.

FIG. 4 shows a simplified schematic diagram of a dosage verification system according to an embodiment of the disclosure.

FIG. 5 shows a flowchart of a method of verifying a dosage of an injection to be administered with a syringe according to an embodiment of the disclosure.

FIG. 6 shows an oblique view of the syringe resting on a flat surface according to an embodiment of the disclosure.

The use of the same reference symbols in different drawings indicates similar or identical items.

### DETAILED DESCRIPTION

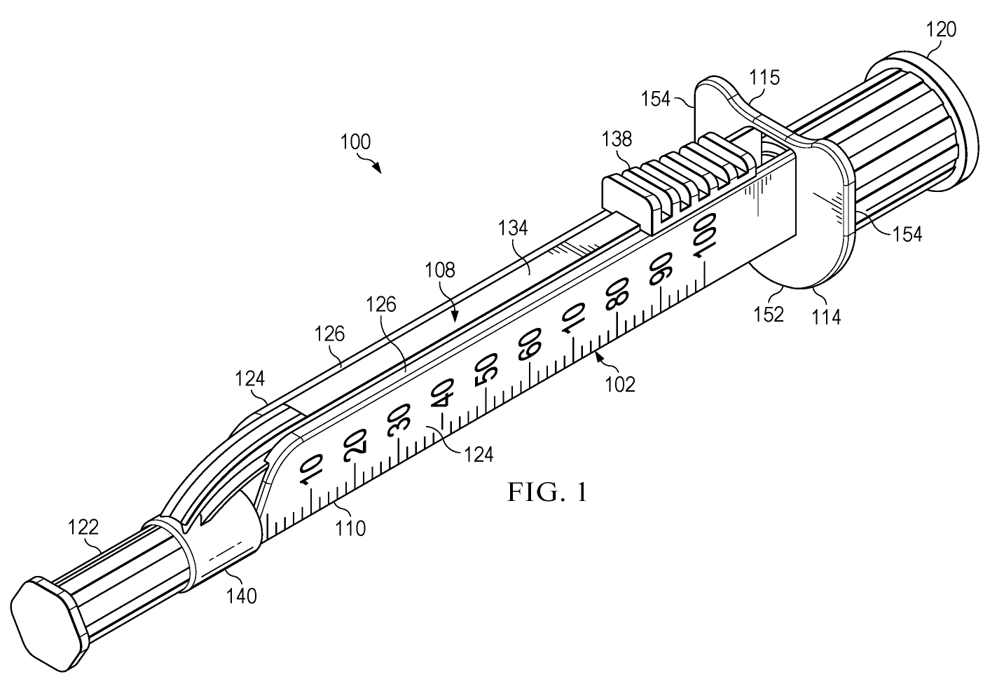

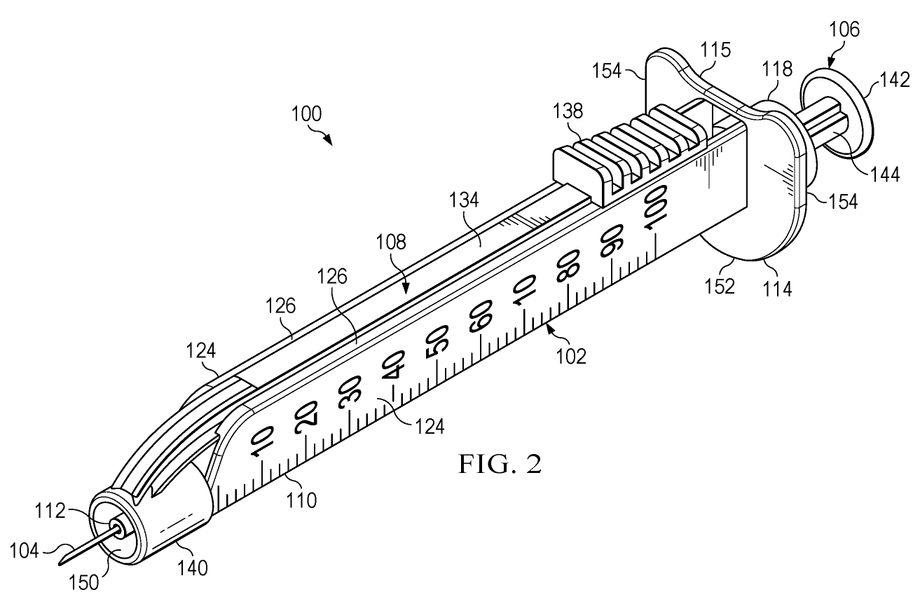

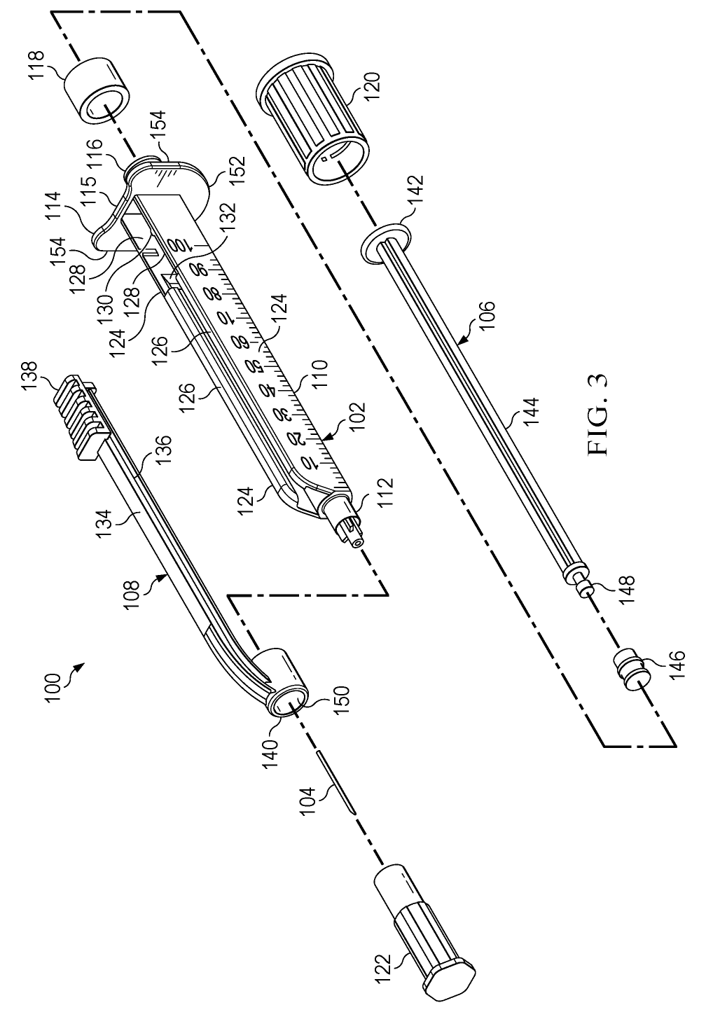

Referring to FIGS. 1-3, an oblique view, another oblique view, and an oblique exploded view of a readable syringe 100 are shown according to an embodiment of the disclosure. Syringe 100 may generally comprise a barrel 102, a needle 104 coupled to the barrel 102, a plunger 106 disposed within the barrel 102, and a needle safety device 108 slidably engaging the barrel 102. Barrel 102 may generally comprise a cylindrical body portion 110 that defines a fluid chamber within the barrel 102. Barrel 102 may comprise a nose 112 disposed at the forward end of the cylindrical body portion 110 for receiving and securing the needle 104 within the barrel 102. Barrel 102 may also comprise an annularly projecting finger flange 114. In some embodiments, the finger flange 114 may comprise a thumb notch 115 on the top side to allow unobstructed access to needle safety device 108.

Barrel 102 may also comprise a cylindrical collar 116 that extends rearwardly from the finger flange 114 and provides a rearwardly facing opening that is coaxially aligned and communicates with the fluid chamber defined by the cylindrical body portion 110 of the barrel 102. In some embodiments, an elastomeric bushing 118 may be disposed around cylindrical collar 116 to releasably secure a plunger cap 120 in place with respect to barrel 102. In some embodiments, a selectively removable needle cap 122 may be installed at least partially within needle safety device 108 and over nose 112 of barrel 102 to cover needle 104 prior to use of syringe 100.

Plunger 106 may generally comprise a plunger surface 142 for selectively actuating the plunger 106 and an elongated plunger body 144 extending therefrom. In some embodiments, a plunger seal 146 may be disposed on or about a smaller diameter projection 148 at a distal end of the elongated plunger body 144. Plunger 106 may be inserted forwardly through rearwardly facing opening of the cylindrical collar 116 and into sliding engagement with fluid chamber of the barrel 102. More specifically, elongated plunger body 144 may be at least partially received within cylindrical body portion 110 of the barrel 102, such that plunger seal 146 forms a fluid tight seal between plunger 106 and fluid chamber defined by cylindrical body portion 110 of barrel 102.

Barrel 102 may comprise a pair of opposing indicia display surfaces 124 that extend from cylindrical body portion 110. Opposing indicia display surfaces 124 may generally be substantially flat and disposed in a parallel relationship. Opposing indicia display surfaces 124 may extend laterally and/or tangentially from cylindrical body portion 110 of barrel 102. In some embodiments, opposing indicia display surfaces 124 may extend forwardly from finger flange 114 to nose 112 of the barrel 102. As such, cylindrical body portion 110, nose 112, finger flange 114, cylindrical collar 116, and opposing indicia display surfaces 124 may be integrally formed in a single manufacturing operation to unitarily form barrel 102. In some embodiments, opposing indicia display surfaces 124 may be free from any markings, including a volumetric scale or other scannable markings used to access or retrieve information about the syringe 100. In some embodiments, one or more of the opposing indicia display surfaces 124 may be marked with a volumetric scale comprising appropriate indicia adjacent to the cylindrical body portion 110 to facilitate easy reading of a liquid level inside fluid chamber of the barrel 102 by a user. Further, in some embodiments, finger flange 114 may comprise a rounded bottom 152 opposite needle safety device 108 and flat, parallel sides 154 that cause syringe 100 to always orient with one of the indicia display surfaces 124 facing upwards from the surface on which it is placed or rests.

Each of opposing indicia display surfaces 124 may comprise a longitudinally extending, inwardly projecting rail 126 disposed on an inner wall 128 of each of the opposing indicia display surfaces 124. Each of opposing indicia display surfaces 124 may also comprise a rear stop 130 and a slide stop 132 disposed rearwardly of the longitudinally extending, inwardly projecting rails 126. Rear stops 130 may be configured to engage needle safety device 108 and retain the needle safety device 108 in a first retracted position where needle tip shield 140 is slid over and surrounds the nose 112 of barrel 102 and tip of the needle 104 is exposed before and during use of the syringe 100, and slide stops 132 may be configured to engage needle safety device 108 and retain the needle safety device 108 in a second extended position where tip of the needle 104 is protected by needle safety device 108 after use of syringe 100.

Needle safety device 108 may comprise a main body 134, a pair of opposing, outwardly facing channels 136 disposed longitudinally along the main body 134, an activation surface 138 disposed on a top of the main body 134 and used to selectively activate the needle safety device 108, and a needle tip shield 140 disposed at a forward end of main body 134. Channels 136 of needle safety device 108 may be cooperatively sized and configured to engage and provide a slidable interface with the longitudinally extending, inwardly projecting rails 126 of opposing indicia display surfaces 124. Following an injection, needle safety device 108 may be selectively activated by applying forward pressure on activation surface 138, which may be angled rearwardly, such that forward motion of a user’s thumb through thumb notch 115 in finger flange 114 may catch activation surface 138 to allow ergonomic sliding movement of needle safety device 108 to disengage needle safety device 108 from rear stops 130. As needle safety device 108 slides forward, slide stops 132 may engage needle safety device 108 and lock the needle safety device 108 in the second extended position. In the second extended position, tip of needle 104 may be disposed within an inner bore 150 of needle tip shield 140, thereby circumferentially surrounding tip of needle 104. Once engaged, slide stops 132 effectively prevent needle safety device 108 from being slid backwards to uncover tip of needle 104, thereby preventing unintended needle sticks or inadvertent contact with needle 104.

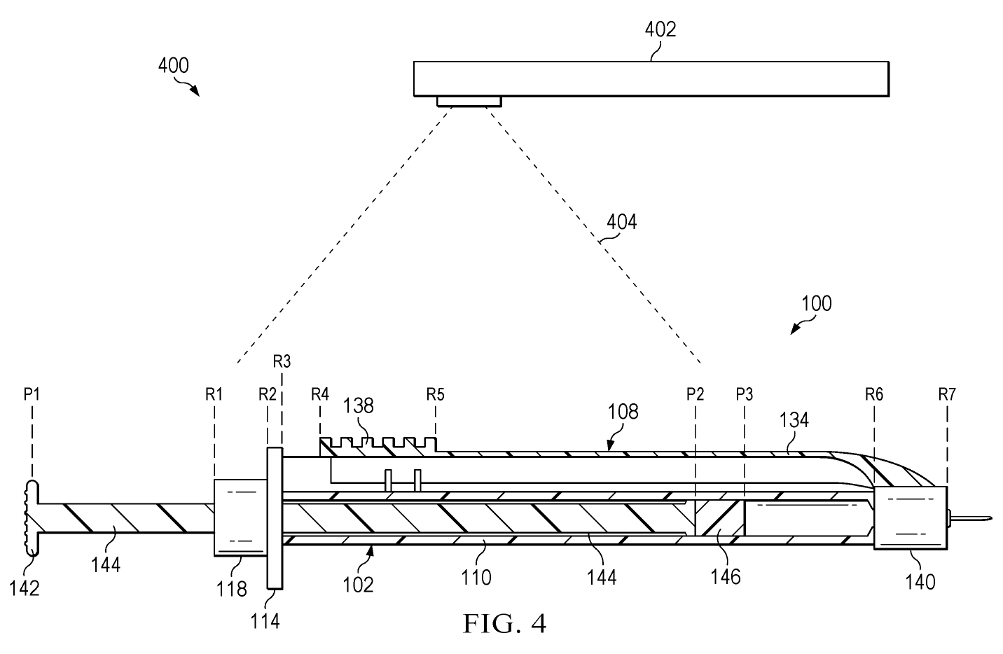

Referring to FIG. 4, a simplified schematic diagram of a dosage verification system 400 is shown according to an embodiment of the disclosure. Dosage verification system 400 may generally comprise a readable syringe 100 to be imaged and an imaging device 402 configured to capture a digital image of syringe 100 and accurately determine a volume of a fluid in syringe 100. Imaging device 402 may generally comprise a camera system configured to capture a digital image of syringe 100, either drawn into syringe 100 or pre-filled, a processing system comprising application software configured to analyze and/or process the captured digital image to accurately determine the volume of the fluid (e.g., dosage) in the syringe 100, and storage/memory capabilities to optionally store the captured digital images and/or other data (e.g., date, time, location, volume of fluid) associated with each captured digital image and/or characteristics or parameters associated with one or more syringes 100. In some embodiments, imaging device 402 may comprise a display screen and/or user interface (e.g., touch screen) that displays the captured image of syringe 100 and/or allows configuration of the application software therethrough. In some embodiments, imaging device 402 may be an application specific device configured for performing methods disclosed herein. In some embodiments, imaging device 402 may be contained in a cell phone, a tablet, and/or a laptop computer.

To capture an image of syringe 100 to verify the volume of fluid therein, imaging device 402 may generally be aimed, directed, or pointed at the syringe 100. Imaging device 402 may generally be configured to be hand-held by a user to capture the digital image of syringe 100, allowing imaging device 402 to be portable and usable in various locations. Additionally, in being a hand-held instrument, the imaging device may be selectively and easily manipulated to capture the digital image of syringe 100. Further, due to the readability of syringe 100, imaging device 402 may be selectively positioned at any angle, distance, and/or orientation with respect to syringe 100 to capture the digital image as long as syringe 100 is within field of view 404 of imaging device 402 and the components of syringe 100 to be imaged are visible by the camera system of the imaging device 402. In some embodiments, entire syringe 100 may not need to be visible by imaging device 402. In some embodiments, the application software in imaging device 402 may be configured to compensate for different orientations of imaging device 402 with respect to syringe 100. In some embodiments, the application software may be configured to compensate for movement of imaging device 402 (or the camera system thereof) during digital image capture and still accurately determine the volume of the fluid in syringe 100.

Imaging device 402 may generally be capable of capturing colored digital images. As such, syringe 100 may generally comprise one or more colored components. In some embodiments, the colored components may aid and/or enable the application software in imaging device 402 to recognize or discern between different components. In some embodiments, the colored components may enhance the accuracy of dosage verification system 400 and/or may enable imaging device 402 and/or the application software to more quickly capture and process the digital image of syringe 100 and/or more accurately determine the volume of fluid in syringe 100 as compared to standard transparent medical syringes or other syringes not having colored components.

Each colored component may comprise a visible RGB color. In some embodiments, each of the colored components may comprise a preferable RGB color selected to maximize the accuracy and/or reliability of dosage verification system 400. In some embodiments, each of the colored components may comprise a preferable RGB color selected to enhance and/or maximize the visual contrast between components. The enhanced or maximized visual contrast between colored components may enable imaging device 402 to easily discern between the colored components in a captured digital image and determine a distance and/or relative position therebetween utilizing known characteristics of syringe 100. Thereafter, the distance and/or relative position between colored components may be used to accurately determine the volume of fluid in syringe 100.

To further enhance or maximize the visual contrast between colored components and/or enhance capabilities of imaging device 402 to prevent imagining errors, corresponding or related colored components of syringe 100 (e.g., components of syringe 100 that move relative to one another and can be used to determine the volume of fluid in the syringe 100) may comprise different colors. In some embodiments, the different colors between related components may be contrasting colors. In some embodiments, the different colors between related components may be complementary or opposite colors (as determined by the RYB color wheel, RGB, or CMYK). For example, if one component is blue (e.g., #76BED0), a related component may be orange (e.g., #ED8F5A). Other color combinations for related components may be red (e.g., #ED5A5A) and green (e.g., #76D095), yellow (e.g., #EDBB5A) and purple (e.g., #D076A1), black and white, and combinations thereof. In a preferred embodiment, plunger 106 may be blue (e.g., #76BED0), needle safety device 108 may be orange (e.g., #ED8F5A), plunger seal 146 may be black, elastomeric bushing 118 may be white, and barrel 102 may be transparent such that plunger 106, needle safety device 108, and the plunger seal 146 are sufficiently visible by imaging device 402 therethrough.

Further, any number of differently colored components may be used. In some embodiments, dosage verification system 400 may utilize more than one set of related components to determine the volume of fluid in syringe 100 and/or may use a backup or secondary set of related components to determine the volume of fluid in syringe 100, as a confirmation of a determined volume of fluid in syringe 100 or as an alternate data source such as where a primary set of related components may not be visible or readable by imaging device 402. Still further, in some embodiments, no two components of syringe 100 that are used in determining the volume of fluid in syringe 100 may comprise the same color.

In some embodiments, a first set of related components usable to determine a volume of fluid in fluid chamber of cylindrical body portion 110 may comprise plunger 106 and needle safety device needle safety device 108. In some embodiments, a second set of related components usable to determine a volume of fluid in fluid chamber of cylindrical body portion 110 may comprise elastomeric bushing 118 and plunger seal 146. In other embodiments, a set of related components usable to determine a volume of fluid in fluid chamber of cylindrical body portion 110 may comprise as a first component plunger 106 or plunger seal 146 and may comprise as a second component needle safety device 108 (or a portion thereof, such as activation surface 138 or needle tip shield 140 or main body 134) or plunger seal 146 or elastomeric bushing 118 or nose 112 or a collar surrounding nose 112. Any combination of first component and second component may be used in any set of related components. In some embodiments, one component in one set of related components may be the same as one component in a different set of related components. In some embodiments, each component in each set of related components is distinct from each other component in the sets of related components.

Preferably, each colored component may generally be molded from a material comprising a single solid color. The color of each colored component may be selected to enhance and/or maximize the visual contrast between it and its related component to maximize the accuracy and/or reliability of dosage verification system 400. Barrel 102 may be molded from a transparent medical grade polymeric material that is sufficiently transparent to permit a user and/or imaging device 402 to see the position of plunger 106 and/or plunger seal 146 inside fluid chamber of cylindrical body portion 110 of barrel 102. In some embodiments, barrel 102 may be partially transparent in an area along a length of fluid chamber of cylindrical body portion 110, such that at least one colored component disposed inside cylindrical body portion 110 is visible to a user or imaging device 402 from a position external to cylindrical body portion 110. Plunger 106 may be molded from a polymeric material. In some embodiments, plunger 106 may be blue and visible through transparent barrel 102. Plunger seal 146 may be molded from an elastomeric material. In some embodiments, plunger seal 146 may be black and be visible through transparent barrel 102 and distinguishable from the plunger 106 by imaging device 402. Elastomeric bushing 118 may be molded from an elastomeric material. In some embodiments, elastomeric bushing 118 may be white and distinguishable from plunger 106 and plunger seal 146 by imaging device 402. However, in alternative embodiments, elastomeric bushing 118 may be black, and elastomeric plunger seal 146 may be white. Needle safety device 108 may be molded from a polymeric material. In some embodiments, needle safety device may be orange and distinguishable from plunger 106 and plunger seal 146 by imaging device 402. However, in alternative embodiments, plunger 106 may be orange, and needle safety device 108 may be blue.

To operate dosage verification system 400, imaging device 402 may be used to capture the digital image of syringe 100. In some embodiments, imaging device 402 may capture the digital image of the entire syringe 100. In some embodiments, imaging device 402 may capture the digital image of only a portion of syringe 100. The captured portion of te syringe 100 may include at least one set of related colored components that move relative to one another based on the volume of the fluid in syringe 100. Accordingly, entire syringe 100 may not need to be visible to imaging device 402 during capture of the digital image, and only related color components of syringe 100 that are used for distance and/or position measurement may be captured.

Imaging device 402 may display an image of syringe 100 prior to or during image capture. In some embodiments, imaging device 402 may generate and/or provide an alert that imaging device 402 needs to be repositioned to capture the digital image of syringe 100. In some embodiments, imaging device 402 may provide the alert when one or more of the related components are not sufficiently visible by imaging device 402 and/or when imaging device 402 needs to be move “closer” to or “farther” from syringe 100 to capture the digital image of syringe 100. The alert may be audible and/or visible on the display or user interface of imaging device 402. Thus, imaging device 402 may reduce and/or prevent user error in capturing the digital image and the digital image of syringe 100 is adequate to accurately verify the dosage of fluid in syringe 100.

In some embodiments, the captured digital images and/or data associated with the digital images (e.g., time, date, location, determined dosage, etc.) may be stored locally on imaging device 402 and/or uploaded to a server, cloud computing system, and/or other database. In some embodiments, imaging device 402 may enable a user to selectively retrieve the data in the future to verify dosages, review treatment history, or the like.

Once the digital image is captured, digital imaging device 402 and/or the processing system may access a database (e.g., either locally from local memory/storage on imaging device 402 and/or remotely from a server, cloud computing system, and/or other database) to identify syringe 100 and/or access information related to syringe 100. In some embodiments, the database may comprise information related to a plurality of different syringes 100. In some embodiments, imaging device 402 may be configured for a single syringe 100, such as an insulin syringe. Further, imaging device 402 may be configured to identify syringe 100 and/or access information related to syringe 100 without scanning a bar code, QR code, or other identifying indicia or markings on or related to the syringe 100, such that imaging device 402 may identify syringe 100 and/or access information related to syringe 100 in response to aiming imaging device 402 at syringe 100 and placing syringe 100 properly within the field of view 404 of imaging device 402. Further, imaging device 402 may generate and/or provide an alert that syringe 100 is not recognized. The alert may be audible and/or visible on the display or user interface of the imaging device 402.

Imaging device 402 may be configured to accurately determine the volume of the fluid (e.g., dosage) in the syringe in syringe 100. In some embodiments, imaging device 402 may be configured to determine the volume of fluid in syringe 100 based on known characteristics of syringe 100 and the position of and/or distance between one or more related components of syringe 100 captured in the digital image of syringe 100. In some embodiments, imaging device 402 may accurately determine the volume of the fluid in syringe 100 based on a position of plunger 106 and/or plunger seal 146 with respect to barrel 102, one or more components of barrel 102, needle safety device 108, one or more components of needle safety device 108, and/or elastomeric bushing 118 as captured in digital image of syringe 100. In some embodiments, imaging device 402 may calculate a distance between related components of syringe 100 and/or a distance between a component of syringe 100 and a reference point on syringe 100 based on the known characteristics of syringe 100 and the relative positions of related components of syringe 100 as captured in the digital image of syringe 100 to accurately determine the volume of fluid in syringe 100.

For example, plunger 106 may comprise primary reference points P1, P2, and P3. P1 may represent a position of the plunger surface 142, P2 may represent a position of the distal end of the elongated plunger body 144 and/or a proximal end of the plunger seal 146, and P3 may represent a position of the distal end of the plunger seal 146. Related reference points R1, R2, R3, R4, R5, R6, and R7 represent reference points on syringe 100 that do not move with respect to plunger 106 before or during an injection. R1 represents a proximal end of elastomeric bushing 118, and the distance between P1 and R1 may represent a length of plunger 106 that extends from barrel 102. R2 represents a distal end of elastomeric bushing 118 and/or a proximal end of finger flange 114 of barrel 102. R3 represents a distal end of finger flange 114 of barrel 102 and/or a proximal end of cylindrical body portion 110 that defines the fluid chamber of barrel 102. R4 represents a proximal end of needle safety device 108. R5 represents a distal end of activation surface 138 of needle safety device 108. R6 represents a distal end of cylindrical body portion 110 that defines the fluid chamber of barrel 102 and/or the proximal end of needle tip shield 140 of needle safety device 108. R7 represents a distal end of needle safety device 108 and/or the distal end of needle tip shield 140 of needle safety device 108. Each of P1 to P3 may be used with respect to any of the related reference points R1 to R7 to determine the volume of fluid in syringe 100.

The imaging device 402 may determine the volume of the fluid in syringe 100 based solely on known characteristics of syringe 100 and the relative positions between related components of syringe 100 as captured in the digital image of syringe 100. Imaging device 402 may determine the volume of the fluid in syringe 100 without scanning and/or reading a bar code, QR code, and/or other identifying indicia or markings on syringe 100, a medicine vial or container, or another object that provides information related to syringe 100 and/or the fluid being injected. Imaging device 402 may also accurately determine the volume of the fluid in syringe 100 with our without numerical indicia on barrel 102 of syringe 100.

Once the volume of fluid is determined, imaging device 402 may be configured to generate and/or provide an alert once the volume of fluid in syringe 100 is determined. In some embodiments, imaging device 402 may audibly dictate or state the determined volume of fluid in syringe 100 and/or display the volume of fluid in syringe 100 on the display or user interface of imaging device 402. In some embodiments, imaging device 402 may audibly provide instructions for the injection to the user and/or display instructions to the user on the display or user interface of imaging device 402. In some embodiments, imaging device 402 may be configured to alert a user if the determined volume of fluid in syringe 100 is in accordance with a dosage schedule or predetermined dosage required by a treatment or injection plan. In some embodiments, the alert may be audible and/or visible on the display or user interface of imaging device 402.

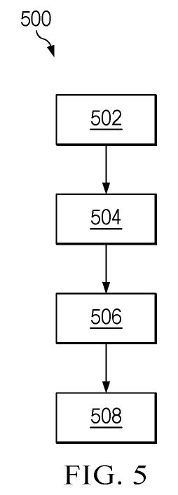

FIG. 5 shows a flowchart of a method 500 of determining a dosage of fluid in a syringe 100 according to an embodiment of the disclosure. Method 500 may begin at block 502 by providing a dosage verification system 400 comprising an imaging device 402 and a readable syringe 100 as disclosed herein. In some embodiments, syringe 100 may comprise a barrel 102, a needle 104 extending from the barrel 102, a plunger 106 disposed within the barrel 102, and a needle safety device 108 slidably engaging the barrel 102. In some embodiments, a fluid may be drawn into the syringe 100 by a user. However, in some embodiments, the fluid may be pre-filled in syringe 100. Method 500 may continue at block 504 by capturing a digital image of syringe 100 with imaging device 402. Method 500 may continue at block 506 by determining the volume of fluid in syringe 100 by imaging device 402. In some embodiments, imaging device 402 may be configured to determine the volume of fluid in syringe 100 based on known characteristics of syringe 100 and the position of and/or distance between one or more related colored components of syringe 100 captured in the digital image of the syringe 100 as disclosed herein. Method 500 may continue at block 508 by preventing subsequent use of syringe 100. In some embodiments, preventing subsequent use of syringe 100 may be accomplished by sliding needle safety device 108 along the barrel 102 to a safe position. In some embodiments, preventing subsequent use of syringe 100 may be accomplished by sliding needle safety device 108 along the barrel 102 to the safe position until the needle safety device 108 engages slide stops 132 and prevents subsequent rearward movement of the needle safety device 108 with respect to the barrel 102.

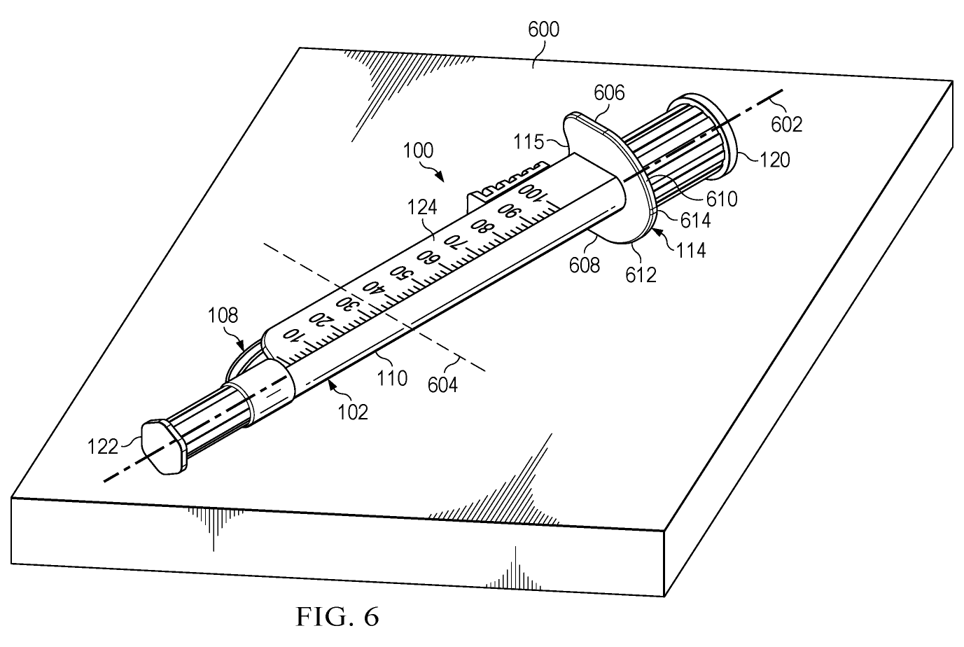

FIG. 6 shows an oblique view of the readable syringe 100 resting on a flat surface 600 according to an embodiment of the disclosure. The syringe 100 comprises the barrel 102, which comprises the cylindrical body portion 110 that defines the fluid chamber within the barrel 102. The pair of opposing indicia display surfaces 124 extend from the barrel 102, are substantially flat, and disposed in a parallel relationship. Each of the indicia display surfaces 124 comprises a longitudinal axis 602 that extends along the barrel 102 and each indicia display surface 124 and a lateral axis 604 that extends across each indicia display surface 124.

Barrel 102 also comprises annularly projecting finger flange 114. Finger flange 114 may generally comprise a first flat edge 606 and an opposing second flat edge 608. Flat edges 608, 610 may generally be disposed in a parallel relationship and oriented in a parallel relationship to the respective indicia display surfaces 124 and/or lateral axis 604. Finger flange 114 may also comprise a first curved edge 610 and a second curved edge 612. First curved edge 610 may connect the first flat edge 606 to the second curved edge 612, and second curved edge 612 may connect the second flat edge 608 to the first curved edge 610. In some embodiments, first curved edge 610 and second curved edge 612 may not meet tangentially, such that the intersection of edges 610, 612 forms a point 614. However, in some embodiments, first curved edge 610 and second curved edge 612 may meet tangentially to form a continuous smooth rounded edge. In some embodiments, finger flange 114 may comprise a thumb notch 115 disposed oppositely from the curved edges 610, 612 on the top side of the finger flange 114 to allow unobstructed access to the needle safety device 108.

In some embodiments, edges 606, 608, 610, 612 are illustrative only, and orienting geometry may instead comprise one or more planar regions, curved regions, chamfered regions, radiused regions, polygonal regions, non-tangential transitions, tangential transitions, discontinuous transitions, or continuously varying surface profiles. The orienting geometry may be symmetric or asymmetric about a longitudinal plane, lateral plane, or other reference plane of syringe 100.

Curved edges 610, 612, may generally be configured to automatically rotationally orient syringe 100 in a most visible reading position when placed on a flat surface 600. In some embodiments, this automatic rotational orientation may enhance the efficiency of capturing an image of syringe 100 and/or enhance the overall use of the dosage verification system 400. When syringe 100 is placed on flat surface 600, curved edges 610, 612 may cooperate to automatically rotate syringe 100, such that one of the flat edges 606, 608 of finger flange 114 will rest on the flat surface 600 and orient syringe 100 such that the respective indicia display surface 124 faces upward. When first curved edge 610 contacts flat surface 600, the arc will cause syringe 100 to rotate until first flat edge 606 rests against the flat surface 600, and when second curved edge 612 contacts flat surface 600, the arc will cause the syringe 100 to rotate until second flat edge 608 rests against flat surface 600, both of which will stop rotation of syringe 100 and stabilize syringe 100 on the flat surface 600. Because one of the flat edges 606, 608 of finger flange 114 rests flat against the flat surface 600, the lateral axis 604 of the upwardly facing indicia display surface 124 will be parallel to the flat surface 600, thereby rotationally orienting syringe 100 with the markings on barrel 102 facing upwardly and in the most visible reading position for a user.

In some embodiments, the orientation feature may be provided by one or more external surfaces, contours, flats, lobes, ribs, rails, edges, arcuate portions, faceted portions, weighted portions, or combinations thereof disposed on finger flange 114, barrel 102, plunger cap 120, needle cap 122, needle safety device 108, or another portion of syringe 100. Such features may cooperate with a support surface to passively bias, urge, or rotationally settle syringe 100 into a predetermined resting orientation, such as an orientation in which one indicia display surface 124 is upwardly facing, outwardly facing, camera-facing, user-facing, substantially visible, more readable, or positioned within a preferred viewing angle for visual inspection or image capture.

In some embodiments, automatic rotational orientation need not occur solely by rolling contact between curved edges and the flat surface. For example, syringe 100 may self-orient based on asymmetrical geometry, differential mass distribution, localized weighting, surface friction, one or more non-circular contact regions, or combinations thereof.

In some embodiments, the structures described herein for orienting syringe 100 on a support surface are illustrative and non-limiting. The orienting function may be achieved by one or more flats, curved portions, faceted portions, chamfers, radiused portions, ribs, protrusions, feet, weighted portions, asymmetric surface contours, or combinations thereof disposed on finger flange 114, barrel 102, plunger cap 120, needle cap 122, needle safety device 108, or another external portion of syringe 100. Such structures may cooperate with a support surface to reduce rolling and rotational ambiguity and to passively bias syringe 100 toward one or more stable resting orientations that improve visibility of indicia, readability of fluid level, consistency of image capture, or detectability of one or more syringe components by imaging device 402. The resulting orientation may be fully upward facing, substantially upward facing, outwardly facing, user-facing, or camera-facing.

As used herein, an “oriented” or “reading” position may include a position in which a selected surface of syringe 100 is fully upward facing, substantially upward facing, laterally facing, outwardly facing, facing an imaging device, facing a user, or otherwise positioned to improve detectability, readability, or image processing accuracy relative to another resting position.

Further, in some embodiments, plunger cap 120 and/or needle cap 122 may comprise edges 606, 608, 610, 612 and function in a similar manner to finger flange 114 to rotationally orient syringe 100 as described herein when one of caps 120, 122 is installed on syringe 100. In some embodiments, plunger cap 120 and/or needle cap 122 may comprise edges 606, 608, 610, 612 to the exclusion of finger flange 114, or alternatively, in addition to finger flange 114. Further, in some embodiments, both of plunger cap 120 and needle cap 122 may comprise edges 606, 608, 610, 612 and cooperate to rotationally orient syringe 100 when both caps 120, 122 are installed on syringe 100. Still further, in other embodiments, a portion of the barrel 102 other than the finger flange 114 may comprise edges 606, 608, 610, 612 to provide the rotational orientation for the syringe 100 as described herein. In some embodiments, barrel 102, finger flange 114, and/or another portion of syringe 100 may comprise one or more weights that operate to aid the orientation of syringe 100.

In some embodiments, an orienting feature may be formed integrally with barrel 102, finger flange 114, plunger cap 120, needle cap 122, needle safety device 108, or another component coupled to syringe 100. In other embodiments, an orienting feature may be provided by an add-on structure, overmolded structure, sleeve, clip, collar, label carrier, cap, shield, cradle-engaging portion, or other attachment. An orienting feature may be permanent, removable, replaceable, or adjustable.

In some embodiments, an orienting feature may orient syringe 100 to one of two or more stable resting positions. In some embodiments, each stable resting position may present a respective indicia display surface 124, a respective machine-readable region, a respective camera target region, or another selected portion of syringe 100 in a preferred viewing orientation.

In some embodiments, the orienting feature may be configured to reduce rolling, reduce rotational ambiguity, improve repeatable placement of syringe 100 on a support surface, improve consistency of image acquisition by imaging device 402, improve user readability of a fluid level or dosage indicia, or combinations thereof.

### EMBODIMENTS

It will be appreciated that embodiments of a syringe 100, a dosage verification system 400, and/or a method 500 of determining a dosage of fluid in a syringe 100 with a dosage verification system 400 may include one or more of the following embodiments:

Embodiment 1. A readable syringe for a dosage verification system, comprising: a transparent barrel comprising a fluid chamber configured to receive a volume of fluid; a first related component that is a first solid color; a second related component that is a second solid color opposite of the first solid color; wherein the first related component moves relative to the second related component; wherein the first related component is distinguishable from the second related component by an imaging device in a digital image captured by the imaging device to determine a position of the first related component with respect to the second related component; and wherein the position of the first related component with respect to the second related component in the digital image is used by the imaging device to determine the volume of fluid in the syringe.

Embodiment 2. The readable syringe of embodiment 1, wherein each solid-colored component is molded from a material comprising a single solid color.

Embodiment 3. The readable syringe of any of the preceding embodiments, wherein the first solid color and the second solid color are visible RGB colors that are contrasting colors.

Embodiment 4. The readable syringe of any of the preceding embodiments, wherein the first solid color and the second solid color are complementary or opposite colors as determined by RYB, RGB, or CMYK color theory.

Embodiment 5. The readable syringe of any of the preceding embodiments, wherein the first related component is a plunger at least partially disposed within the fluid chamber of the barrel, and wherein the second related component is a needle safety device slidably engaged with the barrel.

Embodiment 6. The readable syringe of embodiment 5, wherein the plunger is a solid blue color, and wherein the needle safety device is a solid orange color.

Embodiment 7. The readable syringe of embodiment 6, wherein the plunger is formed from a polymeric material comprising the solid blue color, and wherein the needle safety device is formed from a polymeric material comprising the solid orange color.

Embodiment 8. The readable syringe of any of the preceding embodiments, wherein the first related component is a plunger seal disposed on a plunger and slidably engaging the fluid chamber of the barrel, and wherein the second related component is an elastomeric bushing disposed around a cylindrical collar that extends rearwardly from a finger flange of the barrel.

Embodiment 9. The readable syringe of embodiment 8, wherein the plunger seal is a solid black color, and wherein the elastomeric bushing is a solid white color.

Embodiment 10. The readable syringe of embodiment 9, wherein the plunger seal is formed from an elastomeric material comprising the solid black color, and wherein the elastomeric bushing is formed from an elastomeric material comprising the solid white color.

Embodiment 11. The readable syringe of any of the preceding embodiments, further comprising: a second set of related components used to determine the volume of fluid in the syringe, wherein the second set of related components is used to determine the volume of fluid in the syringe when a primary set of related components comprising the first related component and the second related component are not sufficiently visible or readable by the imaging device.

Embodiment 12. The readable syringe of embodiment 11, wherein no two related components used to determine the volume of fluid in the syringe comprise the same solid color.

Embodiment 13. The readable syringe of any of the preceding embodiments, wherein the imaging device comprises a camera system configured to capture the digital image of the syringe, a processing system comprising application software configured to analyze the captured digital image to determine the volume of the fluid in the syringe, and a user interface that displays the captured digital image of the syringe, the determined volume of fluid in the syringe, or a combination thereof.

Embodiment 14. The readable syringe of embodiment 13, wherein the imaging device is a portable, hand-held imaging device.

Embodiment 15. The readable syringe of any of the preceding embodiments, wherein the imaging device is configured to capture the first related component and the second related component in the digital image to the exclusion of other portions of the syringe.

Embodiment 16. The readable syringe of embodiment 15, wherein the imaging device is configured to generate an alert that the imaging device needs to be repositioned to capture the digital image of the syringe when one or more of the first related component and the second related component are not sufficiently visible by the imaging device.

Embodiment 17. The readable syringe of any of the preceding embodiments, wherein the imaging device is configured to determine the volume of the fluid in the syringe based on known characteristics of the syringe and the position of the first related component with respect to the second related component in the digital image.

Embodiment 18. The readable syringe of embodiment 17, wherein the imaging device is configured to access a database to identify the syringe, access the known characteristics of the syringe, or a combination thereof in response to placing the syringe within a field of view of the imaging device, capturing the digital image of the syringe, or a combination thereof.

Embodiment 19. The readable syringe of any of the preceding embodiments, wherein the imaging device is configured to determine the volume of the fluid in the syringe without scanning or reading a bar code, QR code, or other identifying indicia or markings on the syringe, a medicine vial, or another object that provides information related to the syringe or the fluid in the syringe.

Embodiment 20. The readable syringe of embodiment 19, wherein the imaging device is configured to determine the volume of the fluid in the syringe irrespective of the barrel of the syringe comprising volumetric indicia.

Embodiment 21. The readable syringe of any of the preceding embodiments, wherein the imaging device is configured to generate an alert once the imaging device determines the volume of fluid in the syringe.

Embodiment 22. The readable syringe of embodiment 21, wherein the imaging device is configured to audibly dictate the volume of fluid in the syringe, display the volume of fluid in the syringe, or a combination thereof once the imaging device determines the volume of fluid in the syringe.

Embodiment 23. The readable syringe of embodiment 22, wherein the imaging device is configured to audibly provide instructions for an injection of the volume of the fluid in the syringe, display instructions for the injection of the volume of the fluid in the syringe, or a combination thereof once the imaging device determines the volume of fluid in the syringe.

Embodiment 24. The readable syringe of any of the preceding embodiments, wherein the first related component is a plunger at least partially disposed within the fluid chamber of the barrel, and wherein the first related component is free of markings used by the imaging device to determine the position of the first related component with respect to the second related component, determine the relative position of the first related component to the second related component, determine the volume of fluid in the syringe, or a combination thereof.

Embodiment 25. The readable syringe of embodiment 24, wherein the second related component is not the barrel.

Embodiment 26. The readable syringe of any of the preceding embodiments, wherein the barrel is free of markings used by the imaging device to determine the position of the first related component with respect to the second related component, determine the relative position of the first related component to the second related component, determine the volume of fluid in the syringe, or a combination thereof.

Embodiment 27. A readable syringe for a dosage verification system, comprising: a transparent barrel comprising a cylindrical body portion that defines a fluid chamber configured to receive a volume of fluid within the barrel, a nose disposed at a forward end of the cylindrical body portion, an annularly projecting finger flange disposed around the cylindrical body portion, a pair of substantially flat, parallel opposing indicia display surfaces that extend tangentially from the cylindrical body portion and extend forwardly from the finger flange and longitudinally along the cylindrical body portion proximally to the nose; and a cylindrical collar that extends rearwardly from the finger flange and provides a rearwardly facing opening into the fluid chamber defined by the cylindrical body portion; a plunger received through the rearwardly facing opening and comprising a plunger surface, a plunger body, and a plunger seal slidably engaging the fluid chamber; a needle extending from the nose; an elastomeric bushing disposed around the cylindrical collar; and a needle safety device slidably engaged with the pair of substantially flat, parallel opposing indicia display surfaces; wherein at least one of the plunger and the plunger seal comprise a first related component that is a first solid color; wherein at least one of the needle safety device and the elastomeric bushing comprise a second related component that is a second solid color opposite of the first solid color; wherein the first related component moves relative to the second related component; wherein the first related component is distinguishable from the second related component by an imaging device in a digital image captured by the imaging device to determine a position of the first related component with respect to the second related component; and wherein the position of the first related component with respect to the second related component in the digital image is used by the imaging device to determine the volume of fluid in the syringe.

Embodiment 28. The readable syringe of embodiment 27, wherein each colored component is molded from a material comprising a single solid color.

Embodiment 29. The readable syringe of any of embodiments 27-28, wherein the first solid color and the second solid color are visible RGB colors that are contrasting colors.

Embodiment 30. The readable syringe of any of embodiments 27-29, wherein the first solid color and the second solid color are complementary or opposite colors as determined by RYB, RGB, or CMYK color theory.

Embodiment 31. The readable syringe of any of embodiments 27-30, wherein the first related component is the plunger, and wherein the second related component is the needle safety device.

Embodiment 32. The readable syringe of embodiment 31, wherein the plunger is a solid blue color, and wherein the needle safety device is a solid orange color.

Embodiment 33. The readable syringe of embodiment 32, wherein the plunger is formed from a polymeric material comprising the solid blue color, and wherein the needle safety device is formed from a polymeric material comprising the solid orange color.

Embodiment 34. The readable syringe of any of embodiments 27-30, wherein the first related component is the plunger seal, and wherein the second related component is the elastomeric bushing.

Embodiment 35. The readable syringe of embodiment 34, wherein the plunger seal is a solid black color, and wherein the elastomeric bushing is a solid white color.

Embodiment 36. The readable syringe of embodiment 35, wherein the plunger seal is formed from an elastomeric material comprising the solid black color, and wherein the elastomeric bushing is formed from an elastomeric material comprising the solid white color.

Embodiment 37. The readable syringe of any of embodiments 27-36, further comprising: a second set of related components used to determine the volume of fluid in the syringe, wherein the second set of related components is used to determine the volume of fluid in the syringe when a primary set of related components comprising the first related component and the second related component are not sufficiently visible or readable by the imaging device.

Embodiment 38. The readable syringe of embodiment 37, wherein no two related components used to determine the volume of fluid in the syringe comprise the same solid color.

Embodiment 39. The readable syringe of any of embodiments 27-38, wherein the imaging device comprises a camera system configured to capture the digital image of the syringe, a processing system comprising application software configured to analyze the captured digital image to determine the volume of the fluid in the syringe, and a user interface that displays the captured digital image of the syringe, the determined volume of fluid in the syringe, or a combination thereof.

Embodiment 40. The readable syringe of embodiment 39, wherein the imaging device is a portable, hand-held imaging device.

Embodiment 41. The readable syringe of any of embodiments 27-40, wherein the imaging device is configured to capture the first related component and the second related component in the digital image to the exclusion of other portions of the syringe.

Embodiment 42. The readable syringe of embodiment 41, wherein the imaging device is configured to generate an alert that the imaging device needs to be repositioned to capture the digital image of the syringe when one or more of the first related component and the second related component are not sufficiently visible by the imaging device.

Embodiment 43. The readable syringe of any of embodiments 27-42, wherein the imaging device is configured to determine the volume of the fluid in the syringe based on known characteristics of the syringe and the position of the first related component with respect to the second related component in the digital image.

Embodiment 44. The readable syringe of embodiment 43, wherein the imaging device is configured to access a database to identify the syringe, access the known characteristics of the syringe, or a combination thereof in response to placing the syringe within a field of view of the imaging device, capturing the digital image of the syringe, or a combination thereof.

Embodiment 45. The readable syringe of any of embodiments 27-44, wherein the imaging device is configured to determine the volume of the fluid in the syringe without scanning or reading a bar code, QR code, or other identifying indicia or markings on the syringe, a medicine vial, or another object that provides information related to the syringe or the fluid in the syringe.

Embodiment 46. The readable syringe of embodiment 45, wherein the imaging device is configured to determine the volume of the fluid in the syringe irrespective of the barrel of the syringe comprising volumetric indicia.

Embodiment 47. The readable syringe of any of embodiments 27-46, wherein the imaging device is configured to generate an alert once the imaging device determines the volume of fluid in the syringe.

Embodiment 48. The readable syringe of embodiment 47, wherein the imaging device is configured to audibly dictate the volume of fluid in the syringe, display the volume of fluid in the syringe, or a combination thereof once the imaging device determines the volume of fluid in the syringe.

Embodiment 49. The readable syringe of embodiment 48, wherein the imaging device is configured to audibly provide instructions for an injection of the volume of the fluid in the syringe, display instructions for the injection of the volume of the fluid in the syringe, or a combination thereof once the imaging device determines the volume of fluid in the syringe.

Embodiment 50. A dosage verification system, comprising: a readable syringe according to any of the preceding embodiments; and an imaging device configured to capture a digital image of a first related component and a second related component of the syringe, distinguish the first related component from the second related component in the digital image to determine a position of the first related component with respect to the second related component, and use the position of the first related component with respect to the second related component in the digital image to determine a volume of fluid in the syringe.

Embodiment 51. A method of determining a volume of fluid in a syringe, comprising: providing a dosage verification system comprising an imaging device and a readable syringe according to any of the preceding embodiments; positioning the imaging device relative to the syringe; capturing a digital image of a first related component and a second related component of the syringe; processing the digital image to distinguish the first related component from the second related component in the digital image to determine a position of the first related component with respect to the second related component; and determining the volume of the fluid in the syringe based on the position of the first related component with respect to the second related component in the digital image.

Embodiment 52. The method of embodiment 51, further comprising: determining the volume of the fluid in the syringe based on known characteristics of the syringe and the position of the first related component with respect to the second related component in the digital image.

Embodiment 53. The method of embodiment 52, further comprising: accessing a database to identify the syringe, accessing the known characteristics of the syringe, or a combination thereof in response to placing the syringe within a field of view of the imaging device, capturing the digital image of the syringe, or a combination thereof.

Embodiment 54. The method of embodiment 51, further comprising: determining the volume of the fluid in the syringe without scanning or reading a bar code, QR code, or other identifying indicia or markings on the syringe, a medicine vial, or another object that provides information related to the syringe or the fluid in the syringe.

Embodiment 55. The method of embodiment 54, further comprising: determining the volume of the fluid in the syringe irrespective of the syringe comprising volumetric indicia.

Embodiment 56. The method of embodiment 51, further comprising: generating an alert once the imaging device determines the volume of fluid in the syringe.

Embodiment 57. The method of embodiment 56, further comprising: audibly dictating the volume of fluid in the syringe, displaying the volume of fluid in the syringe, or a combination thereof in response to the imaging device determining the volume of fluid in the syringe.

Embodiment 58. The method of embodiment 57, further comprising: audibly providing instructions for an injection of the volume of the fluid in the syringe, displaying instructions for the injection of the volume of the fluid in the syringe, or a combination thereof in response to the imaging device determining the volume of fluid in the syringe.

Embodiment 59. The method of embodiment 51, further comprising: preventing subsequent use of the syringe by sliding a needle safety device along a barrel of the syringe until the needle safety device engages a pair of slide stops disposed on a pair of substantially flat, parallel opposing indicia display surfaces that prevent subsequent rearward movement of the needle safety device with respect to the barrel.

Embodiment 60. A syringe, comprising: a barrel comprising: a cylindrical body portion that defines a fluid chamber; a pair of substantially flat, opposing indicia display surfaces disposed in parallel and extending longitudinally along the cylindrical body portion; and an annularly projecting finger flange disposed around the cylindrical body portion, wherein the annularly projecting finger flange comprises: a first flat edge disposed in parallel to a first indicia display surface of the pair of substantially flat, opposing indicia display surfaces; a second opposing flat edge disposed in parallel to a second indicia display surface of the pair of substantially flat, opposing indicia display surfaces; a first curved edge joined to the first flat edge; and a second curved edge joined to the second flat edge; wherein the first curved edge and the second curved edge cooperate to automatically rotate the syringe to a position where one of the first flat edge and the second flat edge rest against a flat surface when the syringe is placed on the flat surface such that one of the first curved edge and the second curved edge contacts the flat surface to rotationally orient either the first indicia display surface or the second indicia display surface in an upward facing position.

Embodiment 61. The syringe of embodiment 60, wherein the first curved edge and the second curved edge are not tangential and form a point.

Embodiment 62. The syringe of embodiment 60, wherein the first curved edge and the second curved edge are tangentially aligned to form a continuous smooth rounded edge.

Embodiment 63. The syringe of any of embodiments 60-62, wherein the finger flange comprises a thumb notch disposed oppositely from the first curved edge and the second curved edge on a top side of the finger flange to allow unobstructed access to a needle safety device that slidably engages the first indicia display surface and the second indicia display surface.

Embodiment 64. The syringe of any of embodiments 60-63, wherein the first curved edge is configured to automatically rotate the syringe until the first flat edge rests against the flat surface when the syringe is placed on the flat surface such that the first curved edge initially contacts the flat surface before any other portion of the finger flange.

Embodiment 65. The syringe of embodiment 64, wherein the second curved edge is configured to automatically rotate the syringe until the second flat edge rests against the flat surface when the syringe is placed on the flat surface such that the second curved edge initially contacts the flat surface before any other portion of the finger flange.

Embodiment 66. The syringe of embodiment 65, wherein the first flat edge and the second flat edge stop rotation of the syringe and stabilize the syringe on the flat surface for reading of numeric indicia on the barrel.

Embodiment 67. The syringe of embodiment 66, wherein a lateral axis of the first indicia display surface and the second indicia display surface is disposed parallel to the flat surface when the first flat edge or the second flat edge rests against the flat surface.

Embodiment 68. The syringe of embodiment 67, wherein the numeric indicia on the barrel are facing upwardly in a most visible reading position for a user when the first flat edge or the second flat edge stop rotation of the syringe.

Embodiment 69. The syringe of any of embodiments 60-68, further comprising: a plunger disposed at least partially within the cylindrical body portion and comprising a plunger seal slidably engaging the fluid chamber.

Embodiment 70. The syringe of any of embodiments 60-69, wherein the barrel comprises a nose disposed forwardly of the cylindrical body portion.

Embodiment 71. The syringe of any of embodiments 60-70, further comprising: a needle extending forwardly from the nose.

Embodiment 72. The syringe of any of embodiments 60-71, wherein the cylindrical body portion, the first indicia display surface, the second indicia display surface, and the annularly projecting finger flange are integrally molded to form a one-piece unitarily molded barrel.

Embodiment 73. A syringe, comprising: a barrel comprising: a cylindrical body portion that defines a fluid chamber; a pair of substantially flat, opposing indicia display surfaces disposed in parallel and extending longitudinally along the cylindrical body portion; and an annularly projecting finger flange disposed around the cylindrical body portion, wherein the annularly projecting finger flange comprises: a first flat edge disposed in parallel to a first indicia display surface of the pair of substantially flat, opposing indicia display surfaces; a second flat edge disposed in parallel to a second indicia display surface of the pair of substantially flat, opposing indicia display surfaces; a first curved edge joined to the first flat edge; and a second curved edge joined to the second flat edge; wherein the first curved edge and the second curved edge cooperate to automatically rotate the syringe to a position where one of the first flat edge and the second flat edge rests against a flat surface when the syringe is placed on the flat surface such that either the first indicia display surface or the second indicia display surface is rotationally oriented in an upward facing position.

Embodiment 74. The syringe of embodiment 73, wherein the first curved edge and the second curved edge are non-tangential and cooperate to form a point.

Embodiment 75. The syringe of embodiment 73, wherein the first curved edge and the second curved edge are tangentially aligned to form a continuous rounded edge.

Embodiment 76. The syringe of any one of embodiments 73-75, wherein the annularly projecting finger flange further comprises a thumb notch disposed opposite the first curved edge and the second curved edge on a top side of the annularly projecting finger flange.

Embodiment 77. The syringe of any one of embodiments 73-76, wherein the first curved edge is configured to rotate the syringe until the first flat edge rests against the flat surface when the first curved edge initially contacts the flat surface.

Embodiment 78. The syringe of any one of embodiments 73-77, wherein the second curved edge is configured to rotate the syringe until the second flat edge rests against the flat surface when the second curved edge initially contacts the flat surface.

Embodiment 79. The syringe of any one of embodiments 73-78, wherein the first flat edge and the second flat edge are configured to stop rotation of the syringe and stabilize the syringe on the flat surface.

Embodiment 80. The syringe of any one of embodiments 73-79, wherein a lateral axis of the first indicia display surface and a lateral axis of the second indicia display surface are disposed parallel to the flat surface when the first flat edge or the second flat edge rests against the flat surface.

Embodiment 81. The syringe of any one of embodiments 73-80, wherein numeric indicia on the barrel are oriented upwardly in a visible reading position for a user when the first flat edge or the second flat edge rests against the flat surface.

Embodiment 82. The syringe of any one of embodiments 73-81, further comprising a plunger disposed at least partially within the cylindrical body portion, wherein the plunger comprises a plunger seal slidably engaging the fluid chamber.

Embodiment 83. The syringe of any one of embodiments 73-83, wherein the barrel further comprises a nose disposed forwardly of the cylindrical body portion.

Embodiment 84. The syringe of embodiment 83, further comprising a needle extending forwardly from the nose.

Embodiment 85. The syringe of any one of embodiments 73-84, wherein the cylindrical body portion, the pair of substantially flat, opposing indicia display surfaces, and the annularly projecting finger flange are integrally molded as a one-piece barrel.

Embodiment 86. The syringe of any one of embodiments 73-85, wherein the annularly projecting finger flange comprises one or more orienting surfaces configured to cooperate with the flat surface to bias the syringe toward a stable rotational orientation in which one of the pair of substantially flat, opposing indicia display surfaces faces upwardly.

Embodiment 87. The syringe of embodiment 86, wherein the one or more orienting surfaces comprise one or more curved portions, faceted portions, chamfered portions, radiused portions, or combinations thereof.

Embodiment 88. The syringe of any one of embodiments 86-87, wherein the stable rotational orientation is a preferred viewing orientation for visual inspection of the syringe by a user.

Embodiment 89. The syringe of any one of embodiments 86-87, wherein the stable rotational orientation is a preferred viewing orientation for image capture of the syringe by an imaging device.

Embodiment 90. The syringe of any one of embodiments 73-85, further comprising an orienting feature disposed on a component of the syringe other than the annularly projecting finger flange, wherein the orienting feature is configured to cooperate with the flat surface to bias the syringe toward a stable rotational orientation.

Embodiment 91. The syringe of embodiment 90, wherein the component comprises a plunger cap, a needle cap, or a needle safety device.

Embodiment 92. The syringe of any one of embodiments 90-91, wherein the orienting feature comprises one or more flats, curved portions, ribs, protrusions, feet, or weighted portions.

Embodiment 93. The syringe of any one of embodiments 73-92, wherein the annularly projecting finger flange comprises an asymmetric geometry configured to reduce rotational ambiguity when the syringe is placed on the flat surface.

Embodiment 94. The syringe of any one of embodiments 73-92, wherein the annularly projecting finger flange comprises a weighted region configured to bias the syringe toward a stable resting orientation when the syringe is placed on the flat surface.

Embodiment 95. A readable syringe for a dosage verification system, comprising: a barrel comprising a fluid chamber configured to receive a volume of fluid, wherein at least a portion of the barrel is transparent; a first set of related components comprising a first related component that is a first solid color and a second related component that is a second solid color contrasting with the first solid color; wherein the first related component moves relative to the second related component; wherein the first related component is distinguishable from the second related component by an imaging device in a digital image captured by the imaging device to determine a position of the first related component with respect to the second related component that indicates the volume of fluid in the fluid chamber.

Embodiment 96. The readable syringe of embodiment 96, wherein each solid-colored component is molded from a material comprising a single solid color.

Embodiment 97. The readable syringe of any one of embodiments 95-96, wherein the first solid color and the second solid color are visible RGB colors that are contrasting colors.

Embodiment 98. The readable syringe of any one of embodiments 95-97, wherein the first solid color and the second solid color are complementary or opposite colors as determined by RYB, RGB, or CMYK color theory.

Embodiment 99. The readable syringe of any one of embodiments 95-98, wherein the first related component is a plunger at least partially disposed within the fluid chamber of the barrel, and wherein the second related component is a needle safety device slidably engaged with the barrel.

Embodiment 100. The readable syringe of any one of embodiments 95-99, wherein the first solid color is a solid blue color, and wherein the second solid color is a solid orange color.

Embodiment 101. The readable syringe of any one of embodiments 99-100, wherein the plunger is formed from a polymeric material comprising the solid blue color, and wherein the needle safety device is formed from a polymeric material comprising the solid orange color.

Embodiment 102. The readable syringe of any one of embodiments 95-98, wherein the first related component is a plunger seal disposed on a plunger and slidably engaging the fluid chamber of the barrel, and wherein the second related component is a bushing disposed around a cylindrical collar that extends rearwardly from a finger flange of the barrel.

Embodiment 103. The readable syringe of embodiment 102, wherein the first solid color is a solid black color, and wherein the second solid color is a solid white color.

Embodiment 104. The readable syringe of any one of embodiments 102-103, wherein the plunger seal is formed from an elastomeric material comprising the solid black color, and wherein the bushing is formed from an elastomeric material comprising the solid white color.

Embodiment 105. The readable syringe of any one of embodiments 95-104, further comprising: a second set of related components comprising a third related component that is a third solid color and a fourth related component that is a fourth solid color contrasting with the third solid color; usable to determine the volume of fluid in the fluid chamber wherein the third related component moves relative to the fourth related component; wherein the third related component is distinguishable from the fourth related component by the imaging device in the digital image captured by the imaging device to determine a position of the third related component with respect to the fourth related component that indicates the volume of fluid in the fluid chamber. .

Embodiment 106. The readable syringe of embodiment 105, wherein no two related components used to determine the volume of fluid in the syringe comprise the same solid color.

Embodiment 107. The readable syringe of any one of embodiments 95-106, further comprising the imaging device, wherein the imaging device comprises: a camera system configured to capture the digital image of the readable syringe; a processing system comprising application software configured to analyze the digital image to determine the volume of the fluid in the fluid chamber; and a user interface that displays the digital image of the readable syringe, the volume of fluid in the readable syringe determined by the processing system, or a combination thereof.

Embodiment 108. The readable syringe of embodiment 107, wherein the imaging device is a portable, hand-held imaging device.

Embodiment 109. The readable syringe of any one of embodiments 107-108, wherein the imaging device is configured to capture the first related component and the second related component in the digital image to the exclusion of other portions of the readable syringe.

Embodiment 110. The readable syringe of any one of embodiments 107-109, wherein the imaging device is configured to generate an alert that the imaging device needs to be repositioned to capture the digital image of the syringe when one or more of the first related component and the second related component are not sufficiently visible by the imaging device.

Embodiment 111. The readable syringe of any one of embodiments 107-110, wherein the imaging device is configured to determine the volume of the fluid in the fluid chamber based on known characteristics of the readable syringe and the position of the first related component with respect to the second related component in the digital image.

Embodiment 112. The readable syringe of embodiment 111, wherein the imaging device is configured to access a database to identify the readable syringe, access the known characteristics of the readable syringe, or a combination thereof in response to placing the readable syringe within a field of view of the imaging device, capturing the digital image of the readable syringe, or a combination thereof.

Embodiment 113. The readable syringe of any one of embodiments 107-112, wherein the imaging device is configured to determine the volume of the fluid in the fluid chamber without scanning or reading a bar code, QR code, or other identifying indicia or markings on the readable syringe, a medicine vial, or another object that provides information related to the readable syringe or the fluid in the fluid chamber.

Embodiment 114. The readable syringe of any one of embodiments 107-113, wherein the imaging device is configured to determine the volume of the fluid in the fluid chamber irrespective of the barrel of the syringe comprising volumetric indicia.

Embodiment 115. The readable syringe of any one of embodiments 107-114, wherein the imaging device is configured to generate an alert once the imaging device determines the volume of fluid in the fluid chamber.

Embodiment 116. The readable syringe of any one of embodiments 107-115, wherein the imaging device is configured to audibly dictate the volume of fluid in the fluid chamber, or to visually display a numeric value for the volume of fluid in the fluid chamber, or a combination thereof once the imaging device determines the volume of fluid in the fluid chamber.

Embodiment 117. The readable syringe of any one of embodiments 107-116, wherein the imaging device is configured to audibly provide instructions for an injection of the volume of the fluid in the fluid chamber, or to visibly display instructions for the injection of the volume of the fluid in the fluid chamber, or a combination thereof once the imaging device determines the volume of fluid in the fluid chamber.

Embodiment 118. The readable syringe of any one of embodiments 95-98, 100-101, or 105-117, wherein the first related component is a plunger at least partially disposed within the fluid chamber of the barrel, and wherein the first related component is free of markings usable by the imaging device to determine the position of the first related component with respect to the second related component, or to determine the relative position of the first related component to the second related component, or to determine the volume of fluid in the fluid chamber, or a combination thereof.

Embodiment 119. The readable syringe of any one of embodiments 95-118, wherein the second related component is not the barrel.

Embodiment 120. The readable syringe of any one of embodiments 95-119, wherein the barrel is free of markings usable by the imaging device to determine the position of the first related component with respect to the second related component, or to determine the relative position of the first related component to the second related component, or to determine the volume of fluid in the fluid chamber, or a combination thereof.

Embodiment 121. The readable syringe of any one of embodiments 95-98, 100-101, or 105-120, wherein the first related component comprises a plunger or a plunger seal; wherein the second related component comprises a needle safety device or a portion thereof, or a bushing disposed around a cylindrical collar that extends rearwardly from a finger flange of the barrel, or a nose configured to receive a needle, or a collar surrounding the nose; wherein the third related component comprises the plunger or the plunger seal; and wherein the fourth related component comprises the needle safety device or the portion thereof, the bushing, the nose configured to receive the needle, or the collar surrounding the nose; and wherein (1) the first related component is different from the third related component or (2) the second related component is different from the fourth related component or (3) both.

In the foregoing specification, the concepts have been described with reference to specific embodiments. However, those of ordinary skill in the art appreciate that various modifications and changes can be made without departing from the scope of the invention as set forth in the claims below. Other examples that occur to those skilled in the art are intended to be within the scope of the claims if they have structural elements that do not differ from the literal language of the claims, or if they include equivalent structural elements with insubstantial differences from the literal languages of the claims. Accordingly, the specification and figures are to be regarded in an illustrative rather than in a restrictive sense, and all such modifications are intended to be included within the scope of the invention.

Benefits, other advantages, and solutions to problems have been described above with respect to one or more specific embodiments. After reading the specification, those of ordinary skill in the art will appreciate that certain features that are, for clarity, described herein in the context of separate embodiments, may also be provided in combination in a single embodiment. Conversely, various features that are, for brevity, described in the context of a single embodiment, may also be provided separately or in any subcombination. Further, references to values stated in ranges include each and every value within that range and include sub-ranges that overlap from one preferred range to another preferred range.

Any structural elements or arrangement of structural elements relative to each other or method steps of an embodiment, or a preferred embodiment, herein may be used with any other structural elements, features, components, or steps of other embodiments even if not specifically described with respect to that embodiment, unless such combination is explicitly excluded herein. Any structural element or arrangement of structural elements relative to each other or method steps described as excluded with any particular embodiment, or preferred embodiment, herein may similarly be excluded with any other embodiment herein even if not specifically described with such embodiment. Any structural elements or arrangement of structural elements relative to each other in prior art devices or method steps of prior art methods that are not explicitly disclosed herein may be excluded from embodiments herein.

What is claimed is:

1. A readable syringe for a dosage verification system, comprising:
a barrel comprising a fluid chamber configured to receive a volume of fluid, wherein at least a portion of the barrel is transparent;
a first set of related components comprising a first related component that is a first solid color and a second related component that is a second solid color contrasting with the first solid color;
wherein the first related component moves relative to the second related component;
wherein the first related component is distinguishable from the second related component by an imaging device in a digital image captured by the imaging device to determine a position of the first related component with respect to the second related component that indicates the volume of fluid in the fluid chamber.

2. The readable syringe of claim 1, wherein each solid-colored component is molded from a material comprising a single solid color.

3. The readable syringe of claim 1, wherein the first solid color and the second solid color are visible RGB colors that are contrasting colors.

4. The readable syringe of claim 2, wherein the first solid color and the second solid color are complementary or opposite colors as determined by RYB, RGB, or CMYK color theory.

5. The readable syringe of claim 1, wherein the first related component is a plunger at least partially disposed within the fluid chamber of the barrel, and wherein the second related component is a needle safety device slidably engaged with the barrel.

6. The readable syringe of claim 5, wherein the first solid color is a solid blue color, and wherein the second solid color is a solid orange color.

7. The readable syringe of claim 6, wherein the plunger is formed from a polymeric material comprising the solid blue color, and wherein the needle safety device is formed from a polymeric material comprising the solid orange color.

8. The readable syringe of claim 1, wherein the first related component is a plunger seal disposed on a plunger and slidably engaging the fluid chamber of the barrel, and wherein the second related component is a bushing disposed around a cylindrical collar that extends rearwardly from a finger flange of the barrel.

9. The readable syringe of claim 8, wherein the first solid color is a solid black color, and wherein the second solid color is a solid white color.

10. The readable syringe of claim 9, wherein the plunger seal is formed from an elastomeric material comprising the solid black color, and wherein the bushing is formed from an elastomeric material comprising the solid white color.

11. The readable syringe of claim 1, further comprising: a second set of related components comprising a third related component that is a third solid color and a fourth related component that is a fourth solid color contrasting with the third solid color; usable to determine the volume of fluid in the fluid chamber
wherein the third related component moves relative to the fourth related component;
wherein the third related component is distinguishable from the fourth related component by the imaging device in the digital image captured by the imaging device to determine a position of the third related component with respect to the fourth related component that indicates the volume of fluid in the fluid chamber.

12. The readable syringe of claim 11, wherein no two related components used to determine the volume of fluid in the syringe comprise the same solid color.

13. The readable syringe of claim 1, further comprising the imaging device, wherein the imaging device comprises:
a camera system configured to capture the digital image of the readable syringe;
a processing system comprising application software configured to analyze the digital image to determine the volume of the fluid in the fluid chamber; and
a user interface that displays the digital image of the readable syringe, the volume of fluid in the readable syringe determined by the processing system, or a combination thereof.

14. The readable syringe of claim 13, wherein the imaging device is a portable, hand-held imaging device.

15. The readable syringe of claim 13, wherein the imaging device is configured to capture the first related component and the second related component in the digital image to the exclusion of other portions of the readable syringe.

16. The readable syringe of claim 15, wherein the imaging device is configured to generate an alert that the imaging device needs to be repositioned to capture the digital image of the syringe when one or more of the first related component and the second related component are not sufficiently visible by the imaging device.

17. The readable syringe of claim 13, wherein the imaging device is configured to determine the volume of the fluid in the fluid chamber based on known characteristics of the readable syringe and the position of the first related component with respect to the second related component in the digital image.

18. The readable syringe of claim 17, wherein the imaging device is configured to access a database to identify the readable syringe, access the known characteristics of the readable syringe, or a combination thereof in response to placing the readable syringe within a field of view of the imaging device, capturing the digital image of the readable syringe, or a combination thereof.

19. The readable syringe of claim 13, wherein the imaging device is configured to determine the volume of the fluid in the fluid chamber without scanning or reading a bar code, QR code, or other identifying indicia or markings on the readable syringe, a medicine vial, or another object that provides information related to the readable syringe or the fluid in the fluid chamber.

20. The readable syringe of claim 19, wherein the imaging device is configured to determine the volume of the fluid in the fluid chamber irrespective of the barrel of the syringe comprising volumetric indicia.

21. The readable syringe of claim 13, wherein the imaging device is configured to generate an alert once the imaging device determines the volume of fluid in the fluid chamber.

22. The readable syringe of claim 21, wherein the imaging device is configured to audibly dictate the volume of fluid in the fluid chamber, or to visually display a numeric value for the volume of fluid in the fluid chamber, or a combination thereof once the imaging device determines the volume of fluid in the fluid chamber.

23. The readable syringe of claim 22, wherein the imaging device is configured to audibly provide instructions for an injection of the volume of the fluid in the fluid chamber, or to visibly display instructions for the injection of the volume of the fluid in the fluid chamber, or a combination thereof once the imaging device determines the volume of fluid in the fluid chamber.

24. The readable syringe of claim 1, wherein the first related component is a plunger at least partially disposed within the fluid chamber of the barrel, and wherein the first related component is free of markings usable by the imaging device to determine the position of the first related component with respect to the second related component, determine the volume of fluid in the fluid chamber, or a combination thereof.

25. The readable syringe of claim 24, wherein the second related component is not the barrel.

26. The readable syringe of claim 1, wherein the barrel is free of markings usable by the imaging device to determine the position of the first related component with respect to the second related component, determine the volume of fluid in the fluid chamber, or a combination thereof.

27. The readable syringe of claim 11, wherein the first related component comprises a plunger or a plunger seal;
wherein the second related component comprises a needle safety device or a portion thereof, or a bushing disposed around a cylindrical collar that extends rearwardly from a finger flange of the barrel, or a nose configured to receive a needle, or a collar surrounding the nose;
wherein the third related component comprises the plunger or the plunger seal; and
wherein the fourth related component comprises the needle safety device or the poriton thereof, the bushing disposed around the cylindrical collar that extends rearwardly from the finger flange of the barrel, the nose configured to receive the needle, or the collar surrounding the nose; and
wherein (1) the first related component is different from the third related component or (2) the second related component is different from the fourth related component or (3) both.
